(19) United States
(12) Patent Application Publication
(10) Pub. No.: US 2022/0177932 A1
(43) Pub. Date: Jun. 9, 2022

(54) PROCESS AND COMPOSITION FOR CONTROLLING ETHANOL PRODUCTION

(71) Applicants: Ryan H. Senaratne, Fayetteville, AR (US); Abel Price, Fayetteville, AR (US)

(72) Inventors: Ryan H. Senaratne, Fayetteville, AR (US); Abel Price, Fayetteville, AR (US)

(21) Appl. No.: 17/540,340

(22) Filed: Dec. 2, 2021

Related U.S. Application Data
(60) Provisional application No. 63/122,580, filed on Dec. 8, 2020.

(51) Int. Cl. C12P 7/14; C12N 1/16; C12N 1/20

(52) U.S. Cl. CPC C12P 7/14; C12N 1/16; C12N 1/20; C12N 2500/02; C12N 2500/12; C12N 2500/22; C12N 2500/05; C12N 2500/24; C12N 2500/16; C12N 2500/38

(57) ABSTRACT

The present invention provides a process for controlling the production of ethanol by microbial fermentation of gaseous substrates. More specifically, a process is provided for controlling ethanol productivity through addition of vitamins. In accordance with the process, vitamins B1, B5 and B7 are added in amounts that increase specific ethanol productivity.

This application claims the benefit of U.S. Provisional Application No. 63/122,580, filed Dec. 8, 2020, which is incorporated in its entirety herein by reference.

A process is provided for controlling ethanol productivity through addition of vitamins. More specifically, vitamins B1, B5 and B7 are added in amounts that increase specific ethanol productivity.

### BACKGROUND

Biofuels are important replacements for gasoline. Biofuels include ethanol, which has become a major fuel around the world. Microorganisms can produce ethanol and other compounds from carbon monoxide (CO) through fermentation of gaseous substrates. The CO is often provided to the fermentation as part of a gaseous substrate in the form of a syngas. Gasification of carbonaceous materials to produce producer gas, synthesis gas or syngas that includes carbon monoxide and hydrogen is well known in the art. Typically, such a gasification process involves a partial oxidation or starved-air oxidation of carbonaceous material in which a sub-stoichiometric amount of oxygen is supplied to the gasification process to promote production of carbon monoxide.

Fermentations take place in defined liquid mediums. These mediums will typically include various macro- and micro-nutrient sources that are important in improving fermentation performance. Mediums used in connection with less common substrates, such as gaseous substrates, require well defined mediums to optimize performance. Anaerobic fermentations also require well defined mediums.

U.S. Pat. No. 7,285,402 describes mediums known for use in anaerobic fermentation of gaseous substrates to produce ethanol. Various components and component feed rates in the medium are effective for providing high levels of ethanol productivity. More specifically, U.S. Pat. No. 7,285,402 describes mediums that include thiamine (vitamin B1), pantothenate (vitamin B5) and biotin (vitamin B7). However, U.S. Pat. No. 7,285,402 does not recognize or describe how vitamin combinations and vitamin feed rates can act as a control to regulate culture performance and provide higher volumetric productivity.

U.S. Pat. No. 9,701,987 describes increasing B vitamin concentrations to increase 2,3-butane diol production during fermentations of CO containing substrates. More specifically, U.S. Pat. No. 9,701,987 describes increasing B vitamin concentrations far above cellular requirements to increase 2,3-Butane diol production. However, production of ethanol was not affected. Accordingly, there remains a strong need for processes and medium compositions with optimized B vitamins combination that economically increase specific ethanol productivity and thus improve industry competitiveness.

### SUMMARY

The present invention provides a process for controlling the production of ethanol by microbial fermentation of gaseous substrates. More specifically, the process provides for increasing specific ethanol productivity of gaseous CO fermenting acetogenic bacteria. An increase in the rate of vitamin B5 addition to acetogenic bacteria fermentations increases specific ethanol productivity.

In one aspect, a fermentation process includes providing a CO-containing gaseous substrate to a fermentor that includes a fermentation broth; providing vitamin B1, B5, and B7 to the fermentation broth, wherein a feed rate of vitamin B5 is about 25 to about 150 ug/g cells produced or less; and fermenting the CO-containing gaseous substrate with one or more acetogenic bacteria, wherein the process provides a specific ethanol productivity rate of about 8 g/day/gram cells or more. In another aspect, an amount of vitamin B5 is provided at a feed rate of least 2 times a feed rate of vitamin B7, and the amount of vitamin B5 is provided at a feed rate that is at least 2 times a feed rate of vitamin B1.

In another aspect, a composition includes one or more of a source of NH₄⁺, P, K, Fe, Ni, Co, Se, Zn, W, or Mg; vitamin B1; vitamin B5; and vitamin B7, wherein a feed rate of vitamin B5 is about 25 to about 150 ug/g cells produced or less. In another aspect, an amount of vitamin B5 is provided at a feed rate of least 2 times a feed rate of vitamin B7, and the amount of vitamin B5 is provided at a feed rate that is at least 2 times a feed rate of vitamin B1.

### BRIEF DESCRIPTION OF FIGURES

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 illustrates ethanol productivity in fermentations with Clostridium ljungdalii where Vitamin B7 and Vitamin B1 feeds are held at a lower base level with increasing Vitamin B5 feeds.

FIG. 2 shows ethanol productivity in fermentations with Clostridium ljungdalii lower base levels of vitamin B5 feeds and increasing Vitamin B7 and Vitamin B1 feeds.

FIG. 3 illustrates fermentation with Clostridium authoethanogenum with B7 and B1 feeds held at lower base levels with increasing B5 feeds.

### DETAILED DESCRIPTION

The following description is not to be taken in a limiting sense, but is made merely for the purpose of describing the general principles of exemplary embodiments. The scope of the disclosure should be determined with reference to the claims.

### Definitions

Unless otherwise defined, the following terms as used throughout this specification for the present disclosure are defined as follows and can include either the singular or plural forms of definitions below defined:

The term “about” modifying any amount refers to the variation in that amount encountered in real world conditions, e.g., in the lab, pilot plant, or production facility. For example, an amount of an ingredient or measurement employed in a mixture or quantity when modified by “about” includes the variation and degree of care typically employed in measuring in an experimental condition in production plant or lab. For example, the amount of a component of a product when modified by “about” includes the variation between batches in multiple experiments in the plant or lab and the variation inherent in the analytical method. Whether or not modified by “about,” the amounts include equivalents to those amounts. Any quantity stated herein and modified by “about” can also be employed in the present disclosure as the amount not modified by “about”.

The term “fermentor” includes a fermentation device/bioreactor consisting of one or more vessels and/or towers or piping arrangements, which includes a batch reactor, semi-batch reactor, continuous reactor, continuous stirred tank reactor (CSTR), bubble column reactor, external circulation loop reactor, internal circulation loop reactor, immobilized cell reactor (ICR), trickle bed reactor (TBR), moving bed biofilm reactor (MBBR), gas lift reactor, membrane reactor such as hollow fibre membrane bioreactor (HFMBR), static mixer, gas lift fermentor, or other vessel or other device suitable for gas-liquid contact.

The terms “fermentation”, fermentation process” or “fermentation reaction” and the like are intended to encompass both the growth phase and product biosynthesis phase of the process. In one aspect, fermentation refers to conversion of CO to ethanol.

As used herein, productivity is expressed as specific ethanol productivity in grams of ethanol/day/gram of cells (g/day/gram of cells).

### Control of Specific Ethanol Productivity

The current process utilizes vitamins to control and enhance specific ethanol productivity in fermentation of CO-containing substrates by acetogenic bacteria. In this aspect, the process provides a specific ethanol productivity rate of about 8 g/day/gram of cells or more, in another aspect, a specific ethanol productivity rate of about 10 g/day/gram of cells of cells or more, in another aspect, a specific ethanol productivity rate of about 12 g/day/gram of cells of cells or more, in another aspect, a specific ethanol productivity rate of about 14 g/day/gram of cells of cells or more, in another aspect, a specific ethanol productivity rate of about 8 to about 16 g/day/gram of cells, in another aspect, about 8 to about 14 g/day/gram of cells, in another aspect, about 8 to about 12 g/day/gram of cells, in another aspect, about 10 to about 16 g/day/gram of cells, in another aspect, about 10 to about 14 g/day/gram of cells, and in another aspect about 8 to about 10 g/day/gram of cells.

Vitamin B1, B5 and B7 are provided to the fermentation broth at certain feed rate levels and at certain feed rate levels relative to each other. In this aspect, an amount of vitamin B5 provided is at least about 2 times an amount of vitamin B7, in another aspect, at least about 2.5 times an amount of vitamin B7, in another aspect, at least about 3 times an amount of vitamin B7, in another aspect, at least about 3.5 times an amount of vitamin B7, in another aspect, at least about 4 times an amount of vitamin B7, in another aspect, at least about 4.5 times an amount of vitamin B7, and in another aspect, at least about 5 times an amount of vitamin B7. In another aspect, vitamin B5 provided is at least about 2 times and amount of vitamin B1, in another aspect, at least about 2.5 times an amount of vitamin B1, in another aspect, at least about 3 times an amount of vitamin B1, in another aspect, at least about 3.5 times an amount of vitamin B1, in another aspect, at least about 4 times an amount of vitamin B1, in another aspect, at least about 4.5 times an amount of vitamin B1, and in another aspect, at least about 5 times an amount of vitamin B1.

In another aspect, a feed rate of vitamin B5 to the fermentation broth is maintained at a feed rate of about 150 ug/g of cells produced or less, in another aspect, a feed rate of about 125 ug/g cells produced or less, in another aspect, a feed rate of about 100 ug/g cells produced or less, in another aspect, about 95 ug/g cells produced or less, and in another aspect, about 90 ug/g cells produced or less. Ranges of vitamin B5 may include about 25 to about 150 ug/g of cells produced, in another aspect, about 25 to about 125 ug/g of cells produced, in another aspect, about 25 to about 100 ug/g of cells produced, in another aspect, about 25 to about 90 ug/g of cells produced, in another aspect, about 30 to about 95 ug/g cells produced, in another aspect, about 35 to about 90 ug/g cells produced, in another aspect, about 80 to 150 ug/g cells produced, in another aspect, about 90 to 125 ug/g cells produced, and in another aspect, about 90 to about 100 ug/g cells produced.

In another aspect, a feed rate of vitamin B7 to the fermentation broth is maintained at a feed rate of about 150 ug/g of cells produced or less, in another aspect, a feed rate of about 125 ug/g cells produced or less, in another aspect, a feed rate of about 100 ug/g cells produced or less, in another aspect, about 95 ug/g cells produced or less, in another aspect, about 90 ug/g cells produced or less, in another aspect, about 75 ug/g cells produced or less, in another aspect, about 50 ug/g of cells produced or less, in another aspect, about 30 ug/g of cells produced or less. Ranges of vitamin B7 may include about 5 to about 150 ug/g of cells produced, in another aspect, about 15 to about 150 ug/g of cells produced, in another aspect, about 15 to about 125 ug/g of cells produced, in another aspect, about 15 to about 100 ug/g of cells produced, in another aspect, about 15 to about 90 ug/g of cells produced, in another aspect, about 15 to about 95 ug/g cells produced, in another aspect, about 15 to about 90 ug/g cells produced, in another aspect, about 15 to about 75 ug/g cells produced, in another aspect, about 15 to about 50 ug/g cells produced, and in another aspect, about 15 to about 30 ug/g of cells produced.

In another aspect, a feed rate of vitamin B1 to the fermentation broth is maintained at a feed rate of about 150 ug/g of cells produced or less, in another aspect, a feed rate of about 125 ug/g cells produced or less, in another aspect, a feed rate of about 100 ug/g cells produced or less, in another aspect, about 95 ug/g cells produced or less, and in another aspect, about 90 ug/g cells produced or less. Ranges of vitamin B1 may include about 5 to about 150 ug/g cells produced, in another aspect, 15 to about 150 ug/g of cells produced, in another aspect, about 25 to about 150 ug/g of cells produced, in another aspect, about 25 to about 125 ug/g of cells produced, in another aspect, about 25 to about 100 ug/g of cells produced, in another aspect, about 25 to about 90 ug/g of cells produced, in another aspect, about 30 to about 95 ug/g cells produced, and in another aspect, about 35 to about 90 ug/g cells produced.

### Bioreactor Design and Operation

Descriptions of fermentor designs are described in U.S. Ser. Nos. 13/471,827 and 13/471,858, both filed May 15, 2012, U.S. Ser. No. 13/473,167, filed May 16, 2012, and U.S. Ser. Nos. 16/530,481 and 16/530,502, both filed Aug. 2, 2019, all of which are incorporated herein by reference.

The fermentation should desirably be carried out under appropriate conditions for the desired fermentation to occur (e.g. CO-to-ethanol). Reaction conditions to consider include pressure, temperature, gas flow rate, liquid flow rate, medium pH, agitation rate (if using a stirred tank reactor), inoculum level, and acetic acid concentration to avoid product inhibition. In this aspect, the process includes reaction conditions in the following ranges:
Pressure: about 0 to about 500 psi;
Temperature: about 30° C. to about 42° C.;
Medium pH: about 4 to about 6.9;
Agitation rate: about 100 to about 2000 rpm;
Nutrient supply as described herein.

### CO-Containing Gaseous Substrate

A CO-containing gaseous substrate may include any gas that includes CO. In this aspect, a CO-containing gas may include syngas, industrial gases, and mixtures thereof. In a related aspect, a gaseous substrate may include in addition to CO, nitrogen gas (N₂), carbon dioxide (CO₂), methane gas (CH₄), syngas, and combinations thereof.

Syngas may be provided from any known source. In one aspect, syngas may be sourced from gasification of carbonaceous materials. Gasification involves partial combustion of biomass under a restricted supply of oxygen. The resultant gas may include CO and H₂. In this aspect, syngas will contain at least about 10 mol % CO, in one aspect, at least about 20 mol %, in one aspect, about 10 to about 100 mol %, in another aspect, about 20 to about 100 mol % CO, in another aspect, about 30 to about 90 mol % CO, in another aspect, about 40 to about 80 mol % CO, and in another aspect, about 50 to about 70 mol % CO. Some examples of suitable gasification methods and apparatus are provided in U.S. Ser. Nos. 61/516,667, 61/516,704 and 61/516,646, all of which were filed on Apr. 6, 2011, and in U.S. Ser. Nos. 13/427,144, 13/427,193 and 13/427,247, all of which were filed on Mar. 22, 2012, and all of which are incorporated herein by reference.

In another aspect, the process has applicability to support the production of alcohol from gaseous substrates such as high volume CO-containing industrial gases. In some aspects, a gas that includes CO is derived from carbon containing waste, for example, industrial waste gases or from the gasification of other wastes. As such, the processes represent effective processes for capturing carbon that would otherwise be exhausted into the environment. Examples of industrial gases include gases produced during ferrous metal products manufacturing, non-ferrous products manufacturing, petroleum refining processes, gasification of coal, gasification of biomass, electric power production, carbon black production, ammonia production, methanol production, coke manufacturing and gas reforming.

In another aspect, H₂ may be supplied from industrial waste gases or from the gasification of other wastes. As such, the processes represent effective processes for capturing H₂ that would otherwise be exhausted into the environment. Examples of industrial gases include gases produced during ferrous metal products manufacturing, non-ferrous products manufacturing, petroleum refining processes, gasification of coal, gasification of biomass, electric power production, carbon black production, ammonia production, methanol production and coke manufacturing. Other sources of H₂ may include for example, H₂O electrolysis and bio-generated H₂.

Depending on the composition of the CO-containing substrate, the CO-containing substrate may be provided directly to a fermentation process or may be further modified to include an appropriate H₂ to CO molar ratio. In one aspect, CO-containing substrate provided to the fermentor has an H₂ to CO molar ratio of about 0.2 or more, in another aspect, about 0.25 or more, and in another aspect, about 0.5 or more. In another aspect, CO-containing substrate provided to the fermentor may include about 40 mole percent or more CO plus H₂ and about 30 mole percent or less CO, in another aspect, about 50 mole percent or more CO plus H₂ and about 35 mole percent or less CO, and in another aspect, about 80 mole percent or more CO plus H₂ and about 20 mole percent or less CO.

In one aspect, the CO-containing substrate includes CO and H₂. In this aspect, the CO-containing substrate will contain at least about 10 mol % CO, in one aspect, at least about 20 mol %, in one aspect, about 10 to about 100 mol %, in another aspect, about 20 to about 100 mol % CO, in another aspect, about 30 to about 90 mol % CO, in another aspect, about 40 to about 80 mol % CO, and in another aspect, about 50 to about 70 mol % CO.

Certain gas streams may include a high concentration of CO and low concentrations of H₂. In one aspect, it may be desirable to optimize the composition of the substrate stream in order to achieve higher efficiency of alcohol production and/or overall carbon capture. In another aspect, the concentration of H₂ in the substrate stream may be increased before the stream is passed to the bioreactor.

According to particular aspects of the disclosure, streams from two or more sources can be combined and/or blended to produce a desirable and/or optimized substrate stream. For example, a stream comprising a high concentration of CO, such as the exhaust from a steel mill converter, can be combined with a stream comprising high concentrations of H₂, such as the off-gas from a steel mill coke oven.

Depending on the composition of the gaseous CO-containing substrate, it may also be desirable to treat it to remove any undesired impurities, such as dust particles and chemical impurities such as cyanide, oxygen, before introducing it to the fermentation. For example, the gaseous substrate may be filtered or scrubbed using known methods.

### Acetogenic Bacteria

The process includes conducting fermentations in the fermentation bioreactor with acetogenic bacteria. Examples of useful acetogenic bacteria include those of the genus Clostridium, such as strains of Clostridium ljungdahlii, including those described in WO 2000/68407, EP 117309, U.S. Pat. Nos. 5,173,429, 5,593,886 and 6,368,819, WO 1998/00558 and WO 2002/08438, strains of Clostridium autoethanogenum (DSM 10061 and DSM 19630 of DSMZ, Germany) including those described in WO 2007/117157 and WO 2009/151342, Clostridium ragsdalei (P11, ATCC BAA-622), Clostridium carboxidivorans (ATCC PTA-7827) described in U.S. Patent Application No. 2007/0276447, Clostridium coskatii (ATCC PTA-10522), and Clostridium drakei. Mixed cultures of two or more microorganisms may be used.

### Medium Compositions and Control of Medium Feed Rates

In accordance with one aspect, the fermentation process is started by addition of a suitable medium to the reactor vessel. The liquid contained in the reactor vessel may include any type of suitable nutrient medium or fermentation medium. The nutrient medium will include vitamins and minerals effective for permitting growth of the microorganism being used. Sterilization may not always be required.

In another aspect, concentrations of various medium components for use with acetogenic bacteria are as follows:

| | | Feed Rate |
|---|---|---|
| | Concentration | mg/gram cells |
| Element | mg/L | produced |
| NH₄⁺ | 164-6560 | 41-1640 |
| Fe | 1.7-68 | 0.425-17 |
| Ni | 0.07-2.81 | 0.017-0.702 |
| Co | 0.037-1.49 | 0.009-0.373 |
| Se | 0.027-1.1 | 0.006-0.274 |
| Zn | 0.116-4.64 | 0.198-5.95 |
| W | 0.8-32.1 | 0.26-8.03 |
| K | 39-1573 | 9.83-393.25 |
| Mg | 1.4-57.3 | 0.35-14.32 |
| S | 15-625 | 3.9-156.2 |
| P | 15-601 | 3.76-150.43 |

Process operation maintains a pH in a range of about 4 to about 6.9, in another aspect, about 5 to about 6.5, in another aspect about 5.1 to about 6, and in another aspect, about 5.2 to about 6. The medium includes less than about 0.01 g/L yeast extract and less than about 0.01 g/L carbohydrates.

The composition may include one or more of a source of NH₄⁺, P, K, Fe, Ni, Co, Se, Zn, or Mg. Sources of each of these elements may be as follows.

NH₄⁺: The nitrogen may be provided from a nitrogen source selected from the group consisting of ammonium hydroxide, ammonium chloride, ammonium phosphate, ammonium sulfate, ammonium nitrate, and mixtures thereof.

P: The phosphorous may be provided from a phosphorous source selected from the group consisting of phosphoric acid, ammonium phosphate, potassium phosphate, and mixtures thereof.

K: The potassium may be provided from a potassium source selected from the group consisting of potassium chloride, potassium phosphate, potassium nitrate, potassium sulfate, and mixtures thereof.

Fe: The iron may be provided from an iron source selected from the group consisting of ferrous chloride, ferrous sulfate, and mixtures thereof.

Ni: The nickel may be provided from a nickel source selected from the group consisting of nickel chloride, nickel sulfate, nickel nitrate, and mixtures thereof.

Co: The cobalt may be provided from a cobalt source selected from the group consisting of cobalt chloride, cobalt fluoride, cobalt bromide, cobalt iodide, and mixtures thereof.

Se: The selenium may be provided from Na₂SeO₃, C₃H₆NO₂Se, and mixtures thereof.

Zn: The zinc may be provided from ZnSO₄.

W: The tungsten may be provided from a tungsten source selected from the group consisting of sodium tungstate, calcium tungstate, potassium tungstate, and mixtures thereof.

Mg: The magnesium may be provided from a magnesium source selected from the group consisting of magnesium chloride, magnesium sulfate, magnesium phosphate, and mixtures thereof.

S: The composition may also include sulfur. The sulfur may be provided from a sulfur source selected from the group consisting of cysteine, sodium sulfide, NaHS, NaH₂S and mixtures thereof.

### Fermentation

Upon inoculation, an initial feed gas supply rate is established effective for supplying the initial population of microorganisms. Effluent gas is analyzed to determine the content of the effluent gas. Results of gas analysis are used to control feed gas rates. In this aspect, the process provides a minimal cell density of about 0.1 grams per liter.

In one aspect, nutrients may be added to the culture to increase cell growth rates. Suitable nutrients may include non-carbohydrate fractions of yeast extract.

Upon reaching desired levels, liquid phase and cellular material is withdrawn from the reactor and replenished with medium. The fermentation process is effective for increasing cell density as compared to a starting cell density. In this aspect, the process provides an average cell density of about 2 to about 50 grams/liter, in another aspect, about 2 to about 30 grams/liter, in another aspect, about 2 to about 20 grams/liter, in another aspect, about 2 to about 10 grams/liter, and in another aspect, about 2 to about 6 grams/liter.

### EXAMPLES

### Example 1: Effect of Vitamin Feed Rates

A synthesis gas containing CO, CO₂ and H₂ was continuously introduced into a stirred tank bioreactor containing Clostridium ljungdahlii (Experiments 1-4) or Clostridium authoethanogenum (Experiment 5), along with a liquid medium containing trace metals and salts as described herein. Vitamins were provided using dedicated feed lines.

A New Brunswick Bioflow reactor containing the fermentation medium was started with actively growing Clostridium ljungdahlii (Experiments 1-5) or with Clostridium authoethanogenum (Experiment 6). The rate of agitation of the reactor was set to 800 rpm at the start of the experiment and this agitation rate was maintained throughout the experiment. Feed gas flow to the reactor was increased based on the H₂ and CO uptake of the culture. Temperature in the bioreactor was maintained at about 38° C. throughout the experiment. Samples of gas feed into the bioreactor and off-gas from the bioreactor and fermentation broth in the bioreactor were taken at intervals, for example feed gas, off-gas and fermentation broth were sampled about daily, once two hours and once four hours respectively. Above samples were analyzed for consumption or production of various gas components, broth acetic acid concentration, broth ethanol concentration and the optical density (cell density) of the culture. The unaroused volume of the reactor was maintained between 3000 to 3250 ml throughout the experiment. Further, the gas flow to the reactor was maintained at required gas flow rates by using a mass flow controller. The feed syngas composition was 23% H₂, 35% CO, 29% CO₂ and 13% N₂.

In the following reactor runs, vitamins biotin, thiamine and pantothenate were feed to the reactor using a dedicated stream. Steady state conditions were maintained for a period of time greater than 5 times the cell retention time. Cell mass was essentially replaced 5 times before data collection phase started. After data set was collected, vitamin feed rate was adjusted, adjustment phase was repeated, and next data set was collected. Adjustment phase refers to an amount of time necessary for the culture to equilibrate to a change. In this experiment culture was allowed at least a 3-day adjustment phase. A cell recycle system (CRS) was attached to the reactor before the start of the experiment. During the experiment, medium feed rate was 3.0 to 6.0 ml/min, and through the CRS, 0-5 ml/min permeate was drawn out from the reactor.

The following tables describe the vitamin feed rates and specific ethanol productivity (SEP).

Experiment 1: Pantothenate (B5), Biotin (B7) and Thiamine (B1) feeds are all increased.

| Pantothenate Feed | Biotin Feed | Thiamine Feed | |
|---|---|---|---|
| (μg/g cells | (μg/g cells | (μg/g cells | SEP |
| produced) | produced) | produced) | (g/day/g cells) |
| 23.1 | 18 | 44.6 | 8.07 |
| 42.1 | 32.7 | 81 | 9.8 |
| 64.5 | 50.2 | 124 | 10.7 |

As illustrated in the Table, specific ethanol productivity increased as feed rates of all three vitamins increased.

Experiment 2: Pantothenate (B5), Biotin (B7) and Thiamine (B1) feeds are all increased to higher levels than in Experiment 1.

| Pantothenate Feed | Biotin Feed | Thiamine Feed | |
|---|---|---|---|
| (μg/g cells | (μg/g cells | (μg/g cells | SEP |
| produced) | produced) | produced) | (g/day/g cells) |
| 29.3 | 22.8 | 56.5 | 9.3 |
| 54.9 | 42.7 | 105.7 | 9.9 |
| 81.4 | 63.6 | 156.7 | 11.9 |

As illustrated in the Table, specific ethanol productivity increased as feed rates of all three vitamins increased to higher levels.

Experiment 3: Biotin (B7) and Thiamine (B1) feeds held at a lower base level with increasing Pantothenate (B5) Feeds.

| Pantothenate Feed | Biotin Feed | Thiamine Feed | |
|---|---|---|---|
| (μg/g cells | (μg/g cells | (μg/g cells | SEP |
| produced) | produced) | produced) | (g/day/g cells) |
| 19.33 | 17.76 | 13.36 | 7.95 |
| 37.91 | 17.42 | 13.10 | 8.64 |
| 55.49 | 17.00 | 12.79 | 10.03 |
| 72.34 | 16.62 | 12.50 | 10.33 |
| 108.13 | 19.87 | 14.95 | 11.25 |
| 125.67 | 20.55 | 14.76 | 11.15 |

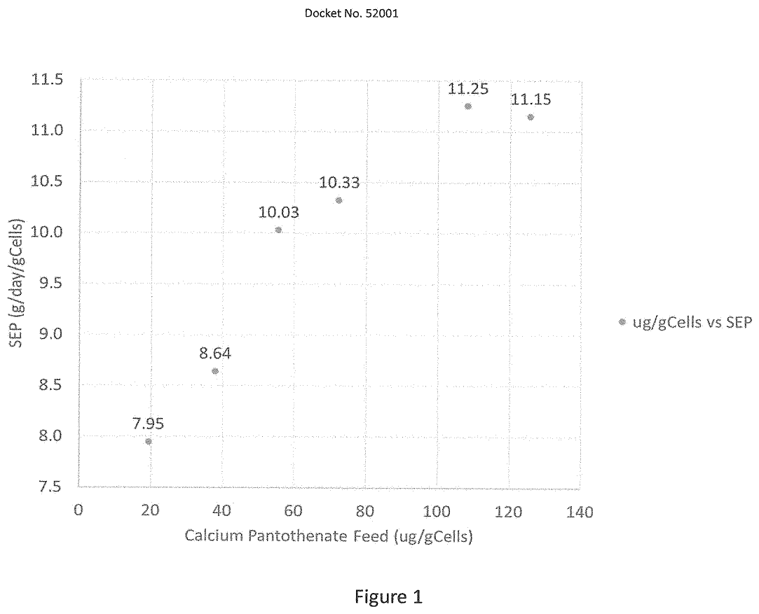

Results of Experiment 3 are illustrated in FIG. 1. By increasing vitamin B5 feed rates from about 20 ug/g of cells produced to about 108 ug/g of cells produced while keeping vitamin B1 and vitamin B7 feed rates under 20 ug/g of cells produced, specific ethanol productivity increase by about 42%.

Experiment 4: Lower base levels of Pantothenate (B5) Feed with increasing Biotin (B7) and Thiamine (B1) Feeds.

| Pantothenate | | Thiamine | | Biotin + |
|---|---|---|---|---|
| Feed | Biotin Feed | Feed | SEP | Thiamine |
| (μg/g cells | (μg/g cells | (μg/g cells | (g/day/ | (μg/g cells |
| produced) | produced) | produced) | g cells) | produced) |
| 29.06 | 26.70 | 20.09 | 7.59 | 46.78 |
| 27.97 | 51.41 | 38.67 | 7.69 | 90.08 |
| 28.41 | 104.42 | 78.56 | 7.31 | 182.98 |

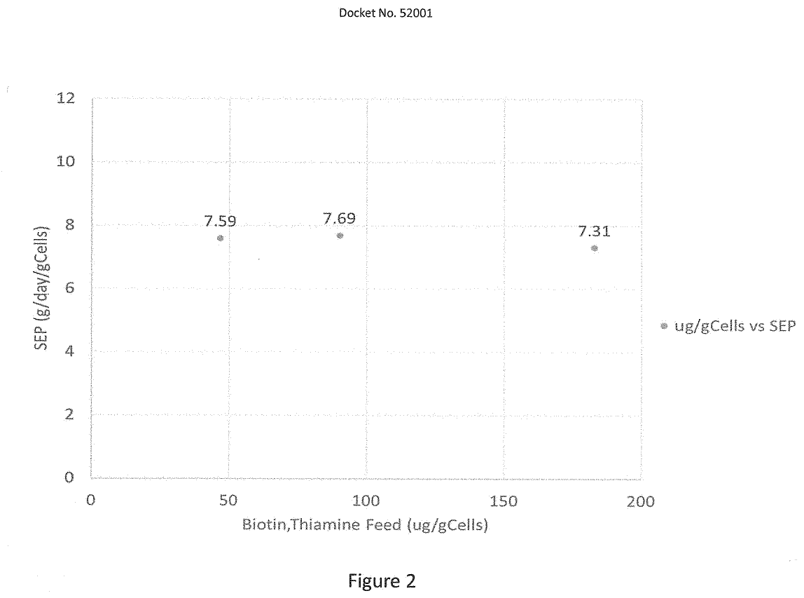

Results of Experiment 4 are illustrated in FIG. 2. Holding vitamin B5 feed rates constant below about 30 ug/g of cells produced while increasing vitamin B1 and vitamin B7 feed rates did not increase specific ethanol productivity.

Experiment 5: Fermentation with Clostridium authoethanogenum with Biotin (B7) and Thiamine (B1) feeds held at lower base levels with increasing Pantothenate (B5) Feeds.

| Pantothenate Feed | Biotin Feed | Thiamine Feed | |
|---|---|---|---|
| (μg/g cells | (μg/g cells | (μg/g cells | SEP |
| produced) | produced) | produced) | (g//day/g cells) |
| 48.47 | 29.54 | 23.10 | 8.08 |
| 58.08 | 26.65 | 20.84 | 8.24 |
| 62.90 | 23.04 | 18.01 | 8.51 |
| 68.78 | 25.19 | 19.70 | 9.39 |
| 70.67 | 21.62 | 16.91 | 9.70 |
| 81.90 | 18.79 | 14.69 | 9.98 |

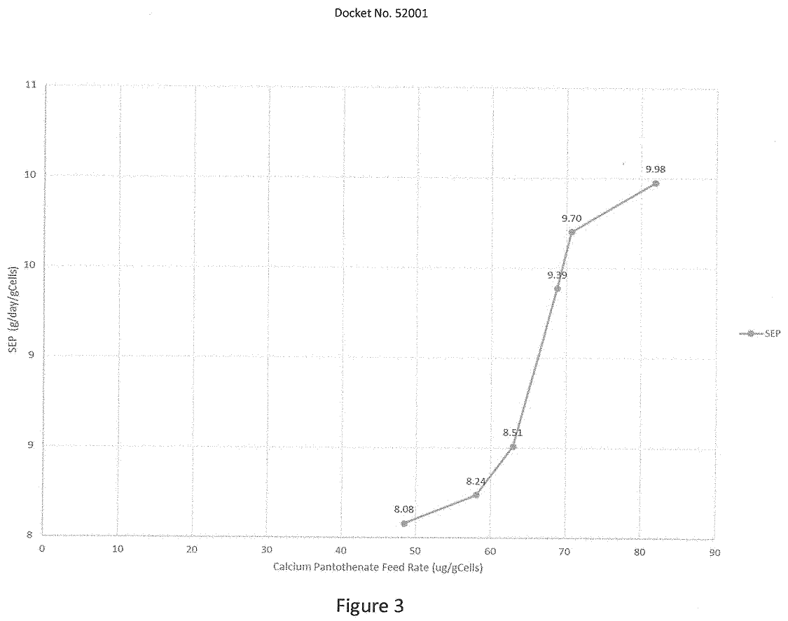

Results of Experiment 5 are illustrated in FIG. 3. By increasing vitamin B5 feed rates from about 48 ug/g of cells produced to about 82 ug/g of cells produced while keeping vitamin B1 and vitamin B7 feed rates under 30 ug/g of cells produced, and further decreasing to under 20 ug/g of cells produced, specific ethanol productivity increase by about 24%.

While the disclosure herein disclosed has been described by means of specific embodiments, examples and applications thereof, numerous modifications and variations could be made thereto by those skilled in the art without departing from the scope of the disclosure set forth in the claims.

What is claimed is:

1. A fermentation process, comprising:
providing a CO-containing gaseous substrate to a fermentor that includes a fermentation broth;
providing vitamin B1, B5, and B7 to the fermentation broth, wherein a feed rate of vitamin B5 is about 25 to about 150 ug/g cell produced or less; and
fermenting the CO-containing gaseous substrate with one or more acetogenic bacteria, wherein the process provides a specific ethanol productivity rate of about 8 g/day/gram cells or more.

2. The fermentation process of claim 1 wherein an amount of vitamin B5 is provided at a feed rate of least 2 times a feed rate of vitamin B7, and the amount of vitamin B5 is provided at a feed rate that is at least 2 times a feed rate of vitamin B1.

3. The fermentation process of claim 1 wherein the acetogenic bacteria is an acetogenic Clostridium.

4. The fermentation process of claim 3 wherein the acetogenic Clostridium is selected from the group consisting of Clostridium ljungdhalii, Clostridium autoethanogum, Clostridium carboxidivorans, Clostridium drakei, Clostridium coskaliii, Clostridium ragsdalei, and mixture thereof.

5. The fermentation process of claim 1 wherein the CO-containing gaseous substrate has a H₂/CO molar ratio of about 0.2 or more.

6. The fermentation process of claim 1 wherein the process provides vitamin B1 to the fermentation broth at a feed rate of less than 100 ug/g cells produced.

7. The fermentation process of claim 1 wherein the process provides vitamin B7 to the fermentation broth at a feed rate of less than 100 ug/g cells produced.

8. The fermentation process of claim 1 wherein the fermentation broth has 0.01 g/L or less yeast extract.

9. The fermentation process of claim 1 wherein the fermentation broth has 0.01 g/L or less carbohydrates.

10. A composition comprising:
one or more of a source of NH₄⁺, P, K, Fe, Ni, Co, Se, Zn, W, or Mg;
vitamin B1;
vitamin B5; and
vitamin B7,
wherein a feed rate of vitamin B5 is about 25 to about 150 ug/g cell produced or less.

11. The composition of claim 10 wherein an amount of vitamin B5 is provided at a feed rate of least 2 times a feed rate of vitamin B7, and the amount of vitamin B5 is provided at a feed rate that is at least 2 times a feed rate of vitamin B1.

12. The composition of claim 10 wherein the composition includes less than about 0.01 grams per liter yeast extract.

13. The composition of claim 10 wherein the composition less than about 0.01 grams per liter carbohydrates.

14. The composition of claim 10 wherein the composition has a pH of about 4 to about 9.

15. The composition of claim 10 wherein the composition comprises:
about 82 to about 3280 mg/L of a NH₄⁺ source;
about 20.12 to about 805 mg/L of a phosphorous source; or
about 98.33 to about 3933 mg/L of a potassium source.

16. The composition of claim 15 wherein the nitrogen is provided from a nitrogen source selected from the group consisting of ammonium hydroxide, ammonium chloride, ammonium phosphate, ammonium sulfate, ammonium nitrate, and mixtures thereof;
the phosphorous is provided from a phosphorous source selected from the group consisting of phosphoric acid, ammonium phosphate, potassium phosphate, and mixtures thereof; and
the potassium is provided from a potassium source selected from the group consisting of potassium chloride, potassium phosphate, potassium nitrate, potassium sulfate, and mixtures thereof.

17. The composition of claim 10 wherein the composition comprises:
about 0.85 to about 34 mg/L of an iron source;
about 0.07 to about 2.81 mg/L of a nickel source;
about 0.037 to about 1.49 mg/L of a cobalt source;
about 0.027 to about 1.1 mg/L of a selenium source;
about 0.59 to about 23.8 mg/L of a zinc source;
about 80.25 to about 3210 mg/L of a tungsten source; or
about 0.71 to about 28.69 mg/L of a magnesium source.

18. The composition of claim 17 wherein the iron is provided from an iron source selected from the group consisting of ferrous chloride, ferrous sulfate, and mixtures thereof;
the nickel is provided from a nickel source selected from the group consisting of nickel chloride, nickel sulfate, nickel nitrate, and mixtures thereof;
the cobalt is provided from a cobalt source selected from the group consisting of cobalt chloride, cobalt fluoride, cobalt bromide, cobalt iodide, and mixtures thereof;
the selenium is provided from a selenium source selected from the group consisting of Na₂SeO₃, C₃H₆NO₂Se, and mixtures thereof;
the zinc is provided from ZnSO₄;
the tungsten is provided from a tungsten source selected from the group consisting of sodium tungstate, calcium tungstate, potassium tungstate, and mixtures thereof; and
the magnesium is provided from a magnesium source selected from the group consisting of magnesium chloride, magnesium sulfate, magnesium phosphate, and the sulfur is provided from a sulfur source selected from the group consisting of cysteine, sodium sulfide, and mixtures thereof.

19. The composition of claim 10 wherein the composition has less than about 100 ug/g cells produced vitamin B1.

20. The composition of claim 10 wherein the composition has less than about 100 ug/g cells produced vitamin B7.
